(19) United States
(12) Patent Application Publication
(10) Pub. No.: US 2011/0089082 A1
(43) Pub. Date: Apr. 21, 2011

(54) Method and device for enhanced metal and amine removal from crude oil with controlled electrostatic coalescence

(75) Inventor: Peter Snawerdt, West Melbourne, FL (US)

(73) Assignee: Assateague Oil llc, West Melbourne, FL (US)

(21) Appl. No.: 12/587,973

(22) Filed: Oct. 15, 2009

(51) Int. Cl. C07C 7/10; C10G 17/04

(52) U.S. Cl. 208/187; 196/14.52

(57) ABSTRACT

A method for removing metals, amines, and other impurities from crude oil in a desalting process that includes the steps of adding a wash water to the crude oil; adding the wash water to the crude oil to create an emulsion; adding to the wash water or the emulsion at least one water-soluble hydroxyacid; selecting the hydroxyacid additive from the group consisting of glycolic acid, gluconic acid, C.sub.2-C.sub.4 alpha-hydroxy acids, malic acid, lactic acid, poly-hydroxy carboxylic acids, thioglycolic acid, chloroacetic acid, polymeric forms of the above hydroxyacids, poly-glycolic esters, glycolate ethers, and ammonium salt and alkali metal salts of these hydroxyacids, and mixtures thereof; resolving the emulsion containing the crude oil, wash water, and hydroxyacid additive into a hydrocarbon phase and an aqueous phase using electrostatic coalescence, the undesired impurities being transferred to the aqueous phase; measuring the characteristics of one or more of the resulting crude oil output, effluent waste, and/or other intermediate points; and altering one or more characteristics of the desalting operation as a function of the measurements. Various oil desalting configurations are also provided.

### FIELD OF THE INVENTION

The present invention relates to a method and device for removing metals, amines, and other contaminants from crude oil and, more particularly, to a method for use in an oil refinery and to an oil refinery for employing such methods.

### BACKGROUND INFORMATION

U.S. Publication No. 2004/0045875 discloses a method for transferring metals and/or amines from a hydrocarbon phase to a water phase in an oil refinery desalting process. The method consists of adding to a wash water an effective amount of a composition comprising certain water-soluble hydroxyacids to transfer metals and/or amines from a hydrocarbon phase to a water phase. The water-soluble hydroxyacid is selected from the group consisting of glycolic acid, gluconic acid, C.sub.2-C.sub.4 alpha-hydroxyacids, malic acid, lactic acid, poly-hydroxy carboxylic acids, thioglycolic acid, chloroacetic acid, polymeric forms of the above hydroxyacids, poly-glycolic esters, glycolate ethers, ammonium salt and alkali metal salts of these hydroxyacids, and mixtures thereof. The pH of the wash water is lowered to 6 or below, before, during and/or after adding the composition and the wash water is added to crude oil to create an emulsion. Finally, the emulsion is resolved into the hydrocarbon phase and an aqueous phase using electrostatic coalescence, where at least a portion of the metals and/or amines are transferred to the aqueous phase.

Optimum Temperature in the Electrostatic Desalting of Maya Crude Oil by Pruneda et al published in the 2005 Journal of the Mexican Chemical Society discloses a simulation model which suggests that there is an optimum temperature to maximize economic benefit when desalting heavy crude oil. As indicated in the art, an increase in process temperature has two effects to be considered. First, as temperature is increased, there is a corresponding decrease in oil density and viscosity which implies a significant increase in the settling rate of water droplets within the oil phase thus allowing a greater amount of oil to be processed resulting in an increase in profit from performing oil desalting. However, crude oil conductivity increases exponentially with temperature which implies a higher rate of electrical power consumption during electrostatic coalescence which increases processing expense.

U.S. Publication No. 2004/0045875 and Optimum Temperature in the Electrostatic Desalting of Maya Crude Oil by Pruneda et al are hereby incorporated by reference herein.

### SUMMARY OF THE INVENTION

U.S. Publication No. 2004/0045875 describes an Electrostatic Desalting Dehydration Apparatus (EDDA) as a laboratory test device, but does not disclose actual electrostatic coalescence in an oil refinery desalting process.

The crude oil temperature, the electric field intensity, the electric voltage waveform used to create the electric field, crude oil feed rate, wash water rate/quality/flow configuration, the control of the water level and emulsion layer, the hydroxyacid addition rate, et al are very important factors that affect refinery desalter performance. As per the present invention, control of these factors to perform electrostatic coalescence when forming the emulsions disclosed in the US 2004/0045875 publication can be especially important.

The present invention provides a method for removing calcium, other metals, and amines from crude oil in a refinery desalting process comprising the steps of:

adding a wash water to the crude oil;

adding the wash water to the crude oil to create an emulsion;

adding to the wash water or the emulsion at least one water-soluble hydroxyacid selected from the group consisting of glycolic acid, gluconic acid, C.sub.2-C.sub.4 alpha-acids, malic acid, lactic acid, poly-hydroxy carboxylic acids, thioglycolic acid, hydroxy chloroacetic acid, polymeric forms of the above hydroxyacids, poly-glycolic esters, glycolate ethers, and ammonium slat and alkali metal salts of these hydroxyacids, and mixtures thereof;

heating at least one of the crude oil, the wash water or the emulsion to a desired temperature;

resolving the emulsion containing the water-soluble hydroxyacid into a hydrocarbon phase and an aqueous phase using electrostatic coalescence, the metals and amines being transferred to the aqueous phase;

measuring a concentration of the metal or amine impurities in the hydrocarbon and/or aqueous phase; and

altering a characteristic of the desalting process to maintain residual impurity levels within the desalted crude as a function of the measured concentration.

The present invention advantageously allows for the adjustment of the crude oil feed rate, the crude oil temperature, the wash water feed rate, the wash water and additive solution mix, the temperature of the oil/wash water emulsion, the electrostatic desalter water level, the addition rate of hydroxyacid additive, and the electric field generated within the electrostatic desalter either individually or in combination as a function of metal and/or amine removal process.

The wash water preferably has a pH of 6 or below and most preferably a pH of 2 to 4.

The present invention also provides a single stage and dual stage electrostatic desalting mechanism applicable to a crude oil refinery. The common elements of each mechanism comprising;

a crude oil supply for storing crude oil;

a wash water supply for supplying wash water to the crude oil to form an emulsion;

a water-soluble hydroxyacid supply for supplying water-soluble hydroxyacid to the wash water or the emulsion;

a heater for changing the temperature of the crude oil, the wash water or the emulsion;

pumps and valves for controlling fluid flow in the desalting process; and

a controller for monitoring, controlling, and varying a characteristic of the desalting operation as a function of the concentration of metal and/or amine impurities in the aqueous phase and/or the hydrocarbon phase.

The characteristic may be, for example, the crude oil feed rate, the crude oil temperature, the wash water feed rate, the wash water and hydroxyacid additive solution mix, the temperature of the oil/wash water emulsion, the electrostatic desalter water level, the addition rate of hydroxyacid additive, the voltage level applied to the electrostatic desalter, the voltage waveform applied to the electrostatic desalter, current limits (if any) on the electrical power supply, or any combination of these.

### BRIEF DESCRIPTION OF THE DRAWINGS

FIG. 1 shows a block diagram of a typical single stage crude oil electrostatic desalting mechanism according to one embodiment of the present invention;

FIG. 2 shows a block diagram of a typical first stage dehydration followed by a second stage electrostatic desalting mechanism according to one embodiment of the present invention;

FIG. 3 shows a block diagram of a typical two stage electrostatic desalting mechanism according to one embodiment of the present invention;

FIG. 4 shows a measurement and control diagram of one embodiment of the method of the present invention for a typical crude oil electrostatic desalting operation;

### DETAILED DESCRIPTION

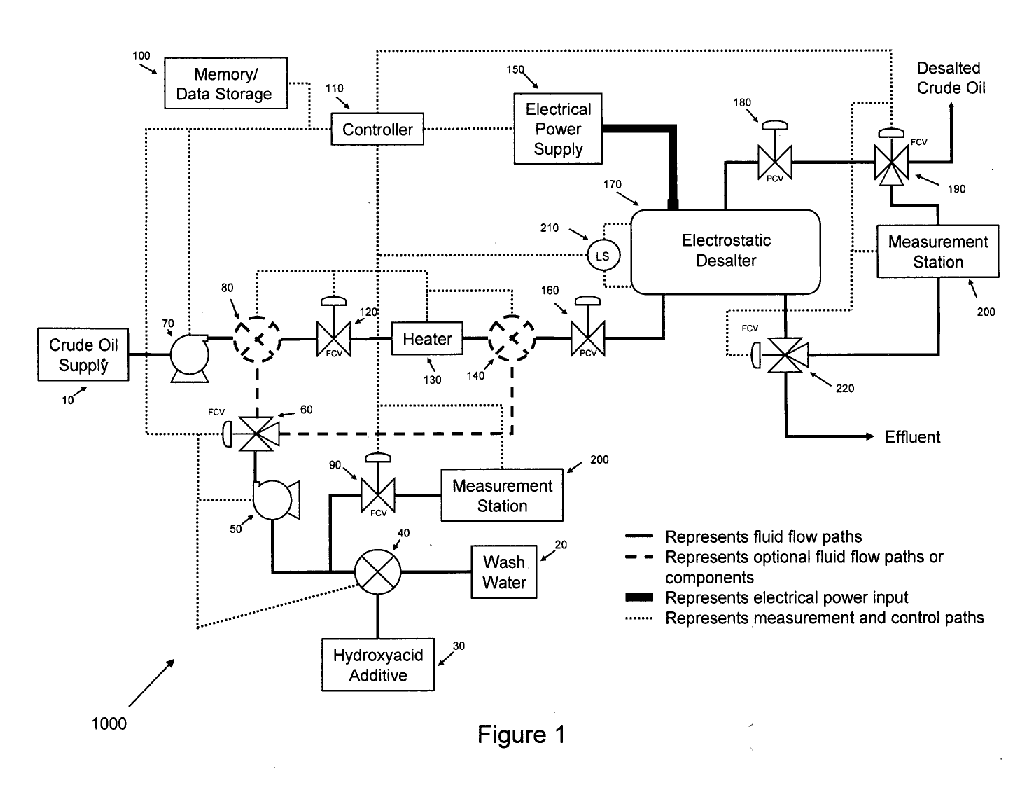

FIG. 1 shows a diagram of a single stage crude oil electrostatic desalting mechanism 1000 of the present invention.

The desalting mechanism 1000 of the present invention includes a crude oil supply 10 for storing crude oil. The crude oil supply 10 is connected to a controllable pump 70 which is connected to an optional controllable fluid mixer 80. The optional controllable fluid mixer 80 allows an emulsion of crude oil 10, wash water 20, and hydroxyacid additive 30 to be created prior to heating based upon the specific characteristics of the crude oil supply 10 to be desalted. The optional controllable fluid mixer 80, if necessary to process the crude oil supply 10, is controlled by the controller 110 to create and maintain the proper emulsion mix of crude oil 10, wash water 20, and hydroxyacid additive 30.

Following either the controllable pump 70 or the optional controllable fluid mixer 80 is a controllable flow control valve (FCV) 120. The controllable flow control valve 120 and the controllable pump 70 work in conjunction under command of the controller 110 to control and maintain the crude oil feed rate and pressure. The crude oil 10 or crude oil emulsion created via optional controllable fluid mixer 80 is then heated to a desired processing temperature by the heater 130 which is controlled by controller 110.

The desalting mechanism 1000 of the present invention also includes a wash water supply 20 and a hydroxyacid additive supply 30 for supplying water-soluble hydroxyacid. In the embodiment of FIG. 1, as is preferred, the hydroxyacid additive 30 is mixed with the wash water 20 by the controllable fluid mixer 40 before the crude oil/wash water emulsion is formed. Alternatively, the hydroxyacid additive 30 could be mixed with the wash water 20 and crude oil 10 during the emulsion creation or after emulsion creation. The fluid mixer 40 is controlled by the controller 110 to create and maintain the proper solution mixture of hydroxyacid additive 30 and wash water 20. The hydroxyacid additive 30 can be selected from the group consisting of glycolic acid, gluconic acid, C.sub.2-C.sub.4 alpha-hydroxy acids, malic acid, lactic acid, poly-hydroxy carboxylic acids, thioglycolic acid, chloroacetic acid, polymeric forms of the above hydroxyacids, poly-glycolic esters, glycolate ethers, and ammonium slat and alkali metal salts of these hydroxyacids, and mixtures thereof. Most preferably, malic acid is used.

After mixing the solution of hydroxyacid additive 30 and wash water 20 with the controllable fluid mixer 40, the resulting solution is input to a controllable flow control valve 90 which is used to allow samples of the mixed hydroxyacid additive 30 and wash water 20 solution to be measured at a measurement station 200. Measurements made on the solution samples would include but not be limited to solution pH, solution impurity levels, and percentage of hydroxyacid additive 30 to wash water 20. This information is sent to the controller 110.

After mixing the solution of hydroxyacid additive 30 and wash water 20 with the controllable fluid mixer 40, the resulting solution is also input to a controllable pump 50 whose output is connected to a controllable flow control valve 60. The controllable pump 50 and the flow control valve 60 work in conjunction under the command of the controller 110 to control and maintain the wash water/hydroxyacid solution feed rate and pressure. In the embodiment of FIG. 1, the controllable flow control valve 60 is shown to be a three-way valve to allow for emulsion creation with the crude oil supply 10 via the optional controllable fluid mixer 80, the optional controllable fluid mixer 140, or both. Like the optional controllable fluid mixer 80, the optional controllable fluid mixer 140, if necessary to process the crude oil supply 10, is controlled by the controller 110 to create and maintain the proper emulsion mix of crude oil 10, wash water 20, and hydroxyacid additive 30. The controllable flow control valve 60 also allows for the hydroxyacid additive 30 and wash water 20 solution to be presented to the optional controllable fluid mixer 80 and optional controllable fluid mixer 140 at the same or different flow rates when both mixer devices are used in the desalting process.

Following the optional controllable fluid mixer 140, the emulsion passes through a pressure control valve 160 before entering the electrostatic desalter 170. The electrostatic desalter 170 includes a liquid level sensor (LS) 210 used to measure the aqueous level in the electrostatic desalter 170. In the embodiment of FIG. 1, the measurement output of the liquid level sensor 210 is routed to the controller 110. The controller 110 uses the liquid level measurement data to control the controllable flow control valve 220 to drain the effluent from the electrostatic desalter 170 and control the aqueous layer and emulsion layer within the electrostatic desalter 170. Alternatively, the liquid level sensor 210 output may be directly connected to a level control valve instead of the controllable flow control valve 220 to drain the effluent. The controllable flow control valve 220 is also configured to allow samples of the effluent solution to be measured at a measurement station 200. Measurements made on the solution samples would include but not be limited to solution pH, solution impurity levels, temperature, and amount of residual oil present in the effluent. This information is sent to the controller 110.

The electrical power supply 150 provides the voltage necessary to create the electric field necessary for electrostatic coalescence in the electrostatic desalter 170. The controller 110 controls the electrical power supply 150 output. The electrical power supply 150 output may be static (i.e. constant voltage with a current limit) or, preferably, able to change key parameters to enhance the desalting operation. The electrical power supply 150 under the control of the controller 110 would preferably be able to alter its' output to include but not be limited to changes in the voltage level applied to the electrostatic desalter 170, the voltage waveform applied to the electrostatic desalter 170, current limits (if any) on the electrical power supply 150, or any combination thereof.

The desalted crude output of the electrostatic desalter 170 passes through a pressure control valve 180 and a controllable flow control valve 190. The controllable flow control valve 190 has two outputs to direct the desalted crude oil. Under control of the controller 110, the controllable flow control valve 190 controls and maintains the flow rate of desalted crude oil to the remaining refinery operations. Additionally, under control of the controller 110, the controllable flow control valve 190 can also direct samples of the desalted crude to the measurement station 200. Measurements made on the solution samples would include but not be limited to impurity levels, temperature, residual hydroxyacid additive 30 and wash water 20 solution, etc. This information is sent to the controller 110.

In the embodiment of FIG. 1, the controller 110 takes measurements including but not limited to the various points described herein to evaluate the efficiency of the desalting mechanism 1000. Based upon the type of crude oil being processed, the controller 110 can adjust various factors of the desalting operation including but not limited to the following:

The crude oil supply 10 feed rate through the controllable pump 70 and controllable flow control valve 120

The temperature of the crude oil supply 10 or, optionally, the emulsion created by mixing the crude oil supply 10 with a solution comprising the hydroxyacid additive 30 and wash water 20 through the controllable heater 130.

The solution mixture of hydroxyacid additive 30 and wash water 20 through the controllable fluid mixer 40.

The flow rate of the solution mixture of hydroxyacid additive 30 and wash water 20 through the controllable pump 50 and controllable flow control valve 60.

The emulsion formation through optional controllable fluid mixer 80 and/or optional controllable fluid mixer 140.

The electrostatic desalter 170 electric field through the controllable electrical power supply 150.

Control of the electrostatic desalter water level and emulsion layers through the liquid level sensor 210, the controllable flow control valve 220, and the controllable flow control valve 190.

As different crude oils are processed by the desalting mechanism 1000, the characteristics necessary to efficiently desalt the crude oil will require some adjustment. Additionally, differences in electrostatic desalter 170 characteristics, wash water supply 20 purity, etc. between different desalting mechanisms 1000 require the storage of different control settings. The memory/data storage 100 function of the desalting mechanism 1000 allows the controller to access and update, if required, the control settings required to efficiently process various types of crude oil supplies 10. Preferably, the control settings are determined based upon maximizing the economic benefit for the desalting the crude oil supply 10. A setpoint residual impurity level for the desalted crude can be determined and the process run to maintain the impurity level below the setpoint.

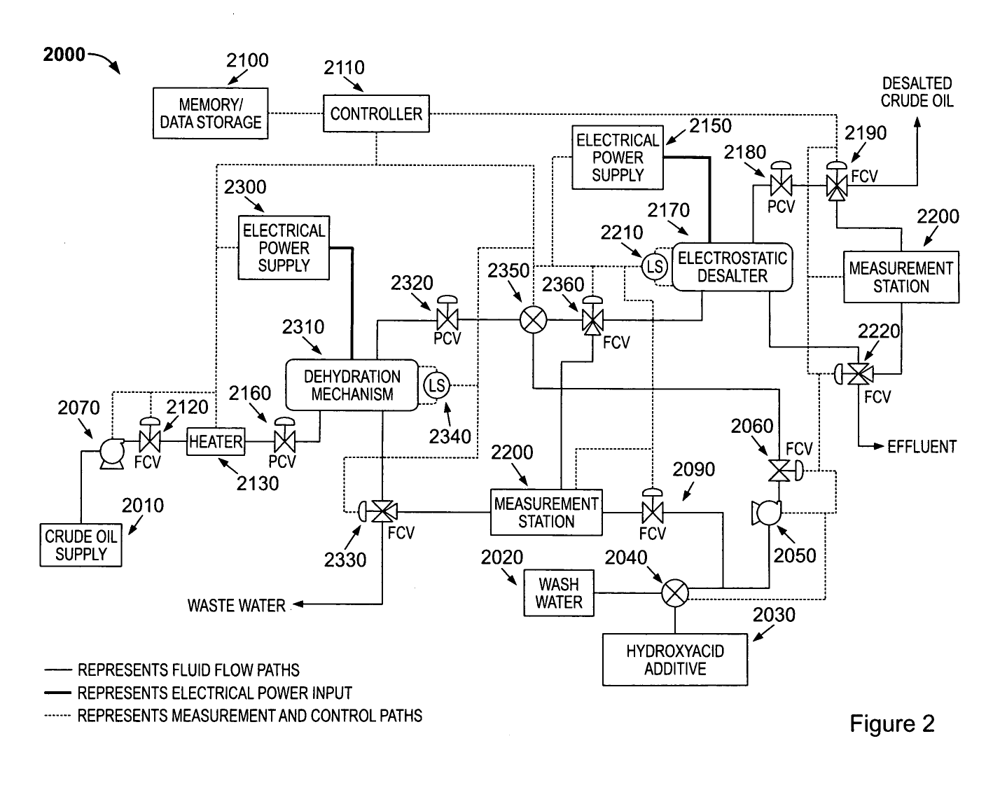

FIG. 2 shows a diagram of a typical first stage dehydration followed by a second stage electrostatic desalting mechanism 2000 of the present invention.

The desalting mechanism 2000 of the present invention includes a crude oil supply 2010 for storing crude oil. The crude oil supply 2010 is connected to a controllable pump 2070 whose output is connected to a controllable flow control valve (FCV) 2120. The controllable flow control valve 2120 and the controllable pump 2070 work in conjunction under command of the controller 2110 to control and maintain the crude oil feed rate and pressure. The crude oil 2010 is then heated to a desired processing temperature by the heater 2130 which is controlled by controller 2110. In the embodiment of FIG. 2, the heated crude oil passes through a pressure control valve 2160 before entering the dehydration mechanism 2310. The dehydration mechanism 2310 is designed to remove high salinity water from the crude oil supply 2010. The dehydration process relies on establishing a varying high voltage electric field in the oil phase of the dehydration mechanism 2310. Due to the action of the imposed electric field, the droplets are agitated causing the drops to coalesce into droplets of sufficient size to migrate via gravity to the lower water phase of the dehydration mechanism 2310. The dehydration mechanism 2310 includes a liquid level sensor (LS) 2340 used to measure the water level in the dehydration mechanism 2310. In the embodiment of FIG. 2, the measurement output of the liquid level sensor 2340 is routed to the controller 2110. The controller 2110 uses the liquid level measurement data to control the controllable flow control valve 2330 to drain the waste water from the dehydration mechanism 2310 and control the water layer and oil layer within the dehydration mechanism 2310. Alternatively, the liquid level sensor 2340 output may be directly connected to a level control valve instead of the controllable flow control valve 2330 to drain the waste water. The controllable flow control valve 2330 is also configured to allow samples of the effluent solution to be measured at a measurement station 2200. Measurements made on the solution samples would include but not be limited to solution pH, solution impurity levels, temperature, and amount of residual oil present in the waste water. This information is sent to the controller 2110.

The electrical power supply 2300 provides the voltage necessary to create the electric field necessary for water coalescence in the dehydration mechanism 2310. The controller 2110 controls the electrical power supply 2300 output. The electrical power supply 2300 output may be static (i.e. constant voltage with a current limit) or, preferably, able to change key parameters to enhance the dehydration operation. The electrical power supply 2300 under the control of the controller 2110 would preferably be able to alter its' output to include but not be limited to changes in the voltage level applied to the dehydration mechanism 2310, the voltage waveform applied to the dehydrator, current limits (if any) on the electrical power supply 2300, or any combination thereof.

The crude output of the dehydration mechanism 2310 passes through a pressure control valve 2320 on its way to the controllable fluid mixer 2350. The controllable fluid mixer 2350 allows an emulsion of crude oil 2010, wash water 2020, and hydroxyacid additive 2030 to be created based upon the specific characteristics of the crude oil supply 2010 to be desalted. The controllable fluid mixer 2350 is controlled by the controller 2110 to create and maintain the proper emulsion mix of crude oil 2010, wash water 2020, and hydroxyacid additive 2030.

The desalting mechanism 2000 of the present invention also includes a wash water supply 2020 and a hydroxyacid additive supply 2030 for supplying water-soluble hydroxyacid. In the embodiment of FIG. 2, as is preferred, the hydroxyacid additive 2030 is mixed with the wash water 2020 by the controllable fluid mixer 2040 before the crude oil/wash water emulsion is formed. Alternatively, the hydroxyacid additive 2030 could be mixed with the wash water 2020 and crude oil 2010 during the emulsion creation or after emulsion creation. The fluid mixer 2040 is controlled by the controller 2110 to create and maintain the proper solution mixture of hydroxyacid additive 2030 and wash water 2020. The hydroxyacid additive 2030 can be selected from the group consisting of glycolic acid, gluconic acid, C.sub.2-C.sub.4 alpha-hydroxy acids, malic acid, lactic acid, poly-hydroxy carboxylic acids, thioglycolic acid, chloroacetic acid, polymeric forms of the above hydroxyacids, poly-glycolic esters, glycolate ethers, and ammonium slat and alkali metal salts of these hydroxyacids, and mixtures thereof.

After mixing the solution of hydroxyacid additive 2030 and wash water 2020 with the controllable fluid mixer 2040, the resulting solution is input to a controllable flow control valve 2090 which is used to allow samples of the mixed hydroxyacid additive 2030 and wash water 2020 solution to be measured at a measurement station 2200. Measurements made on the solution samples would include but not be limited to solution pH, solution impurity levels, and percentage of hydroxyacid additive 2030 to wash water 2020. This information is sent to the controller 2110.

After mixing the solution of hydroxyacid additive 2030 and wash water 2020 with the controllable fluid mixer 2040, the resulting solution is also input to a controllable pump 2050 whose output is connected to a controllable flow control valve 2060. The controllable pump 2050 and the flow control valve 2060 work in conjunction under the command of the controller 2110 to control and maintain the wash water/hydroxyacid solution feed rate and pressure. The output of the flow control valve 2060 is an input to the controllable fluid mixer 2350 where the emulsion of crude oil 2010, wash water 2020, and hydroxyacid additive 2030 is formed.

After mixing the crude oil 2010, hydroxyacid additive 2030, and wash water 2020 in the controllable fluid mixer 2350, the resulting emulsion passes through a controllable flow control valve 2360 before entering the electrostatic desalter 2170. The controllable flow control valve 2360, under command of the controller 2110, controls the flow rate of the crude oil emulsion into the electrostatic desalter 2170 as well as allowing samples of the emulsion to be directed to the measurement station 2200. Measurements made on the solution samples would include but not be limited to impurity levels, temperature, amount of hydroxyacid additive 2030 and wash water 2020 solution, etc. This information is sent to the controller 2110.

The electrostatic desalter 2170 includes a liquid level sensor (LS) 2210 used to measure the aqueous level in the electrostatic desalter 2170. In the embodiment of FIG. 2, the measurement output of the liquid level sensor 2210 is routed to the controller 2110. The controller 2110 uses the liquid level measurement data to control the controllable flow control valve 2220 to drain the effluent from the electrostatic desalter 2170 and control the aqueous layer and emulsion layer within the electrostatic desalter 2170. Alternatively, the liquid level sensor 2210 output may be directly connected to a level control valve instead of the controllable flow control valve 2220 to drain the effluent. The controllable flow control valve 2220 is also configured to allow samples of the effluent solution to be measured at a measurement station 2200. Measurements made on the solution samples would include but not be limited to solution pH, solution impurity levels, temperature, and amount of residual oil present in the effluent. This information is sent to the controller 2110.

The electrical power supply 2150 provides the voltage necessary to create the electric field necessary for electrostatic coalescence in the electrostatic desalter 2170. The controller 2110 controls the electrical power supply 2150 output. The electrical power supply 2150 output may be static (i.e. constant voltage with a current limit) or, preferably, able to change key parameters to enhance the desalting operation. The electrical power supply 2150 under the control of the controller 2110 would preferably be able to alter its' output to include but not be limited to changes in the voltage level applied to the electrostatic desalter 2170, the voltage waveform applied to the electrostatic desalter 2170, current limits (if any) on the electrical power supply 2150, or any combination thereof.

The desalted crude output of the electrostatic desalter 2170 passes through a pressure control valve 2180 and a controllable flow control valve 2190. The controllable flow control valve 2190 has two outputs to direct the desalted crude oil. Under control of the controller 2110, the controllable flow control valve 2190 controls and maintains the flow rate of desalted crude oil to the remaining refinery operations. Additionally, under control of the controller 2110, the controllable flow control valve 2190 can also direct samples of the desalted crude to the measurement station 2200. Measurements made on the solution samples would include but not be limited to impurity levels, temperature, residual hydroxyacid additive 2030 and wash water 2020 solution, etc. This information is sent to the controller 2110.

In the embodiment of FIG. 2, the controller 2110 takes measurements including but not limited to the various points described herein to evaluate the efficiency of the desalting mechanism 2000. Based upon the type of crude oil being processed, the controller 2110 can adjust various factors of the desalting operation including but not limited to the following:

The crude oil supply 2010 feed rate through the controllable pump 2070 and controllable flow control valve 2120

The temperature of the crude oil supply 2010 through the controllable heater 2130.

Control of the dehydration mechanism 2310 water level and oil layers through the liquid level sensor 2340, the controllable flow control valve 2330, and the controllable flow control valve 2360.

The dehydration mechanism 2310 electric field through the controllable power supply 2300.

The solution mixture of hydroxyacid additive 2030 and wash water 2020 through the controllable fluid mixer 2040.

The flow rate of the solution mixture of hydroxyacid additive 2030 and wash water 2020 through the controllable pump 2050 and controllable flow control valve 2060.

The emulsion formation through controllable fluid mixer 2350.

The electrostatic desalter 2170 electric field through the controllable electrical power supply 2150.

Control of the electrostatic desalter 2170 water level and emulsion layers through the liquid level sensor 2210, the controllable flow control valve 2220, and the controllable flow control valve 2190.

As different crude oils are processed by the desalting mechanism 2000, the characteristics necessary to efficiently desalt the crude oil will require some adjustment. Additionally, differences in dehydration mechanism 2310 characteristics, electrostatic desalter 2170 characteristics, wash water supply 2020 purity, etc between different desalting mechanisms 2000 require the storage of different control settings. The memory/data storage 2100 function of the desalting mechanism 2000 allows the controller to access and update, if required, the control settings required to efficiently process various types of crude oil supplies 2010. Preferably, the control settings are determined based upon maximizing the economic benefit for the desalting the crude oil supply 2010.

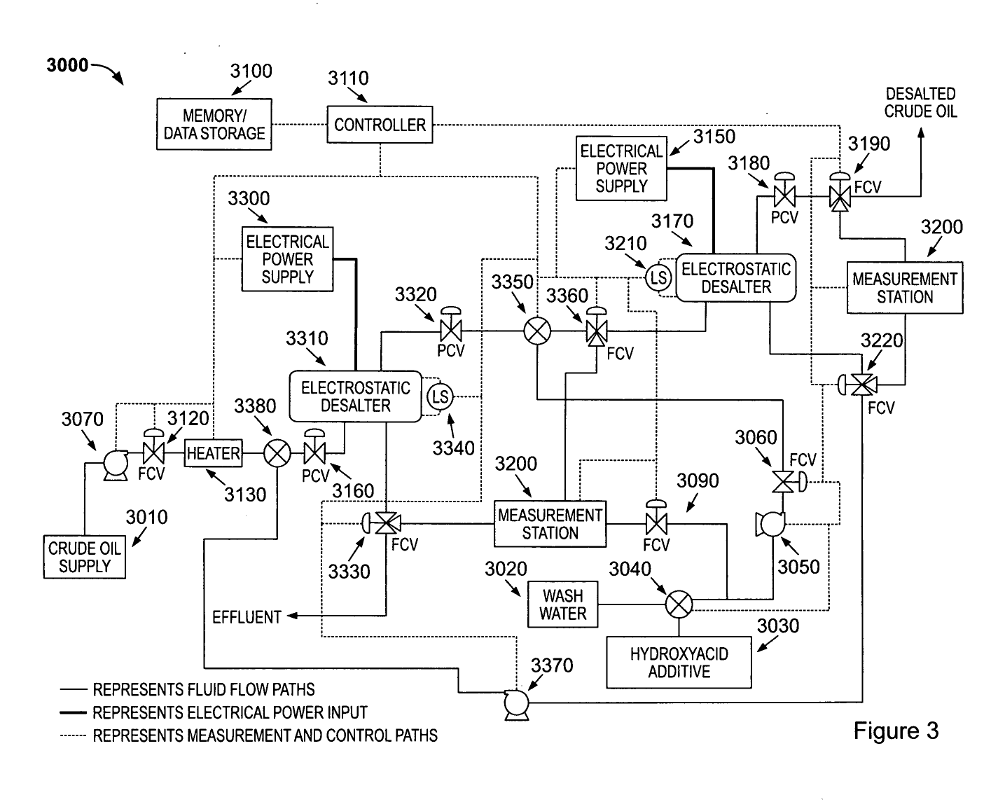

FIG. 3 shows a diagram of a typical two stage electrostatic desalting mechanism 3000 of the present invention.

The desalting mechanism 3000 of the present invention includes a crude oil supply 3010 for storing crude oil. The crude oil supply 3010 is connected to a controllable pump 3070 whose output is connected to a controllable flow control valve (FCV) 3120. The controllable flow control valve 3120 and the controllable pump 3070 work in conjunction under command of the controller 3110 to control and maintain the crude oil feed rate and pressure. The crude oil 3010 is heated to a desired processing temperature by the heater 3130 which is controlled by controller 3110. In the embodiment of FIG. 3, the heated crude oil is mixed with recycled effluent from the electrostatic desalter 3170 to create an emulsion mix of the crude oil supply 3010 and recycled effluent from the electrostatic deslater 3170 via the controllable fluid mixer 3380. Use of an effluent recycle as indicated in FIG. 3 is well-known in the art. The crude oil/effluent recycle emulsion passes through a pressure control valve 3160 before entering the electrostatic desalter 3310. The electrostatic desalter 3310 includes a liquid level sensor (LS) 3340 used to measure the aqueous level in the electrostatic desalter 3310. In the embodiment of FIG. 3, the measurement output of the liquid level sensor 3340 is routed to the controller 3110. The controller 3110 uses the liquid level measurement data to control the controllable flow control valve 3330 to drain the waste effluent from the electrostatic desalter 3310 and control the aqueous layer and emulsion layer within the electrostatic desalter 3310. Alternatively, the liquid level sensor 3340 output may be directly connected to a level control valve instead of the controllable flow control valve 3330 to drain the waste effluent. The controllable flow control valve 3330 is also configured to allow samples of the waste effluent solution to be measured at a measurement station 3200. Measurements made on the solution samples would include but not be limited to solution pH, solution impurity levels, temperature, and amount of residual oil present in the waste effluent. This information is sent to the controller 3110.

The electrical power supply 3300 provides the voltage necessary to create the electric field necessary for electrostatic coalescence in the electrostatic desalter 3310. The controller 3110 controls the electrical power supply 3300 output. The electrical power supply 3300 output may be static (i.e. constant voltage with a current limit) or, preferably, able to change key parameters to enhance the electrostatic coalescence operation. The electrical power supply 3300 under the control of the controller 3110 would preferably be able to alter its' output to include but not be limited to changes in the voltage level applied to the electrostatic desalter 3310, the voltage waveform applied to the desalter, current limits (if any) on the electrical power supply 3300, or any combination thereof.

The crude output of the electrostatic desalter 3310 passes through a pressure control valve 3320 on its way to the controllable fluid mixer 3350. The controllable fluid mixer 3350 allows a second emulsion of electrostatic desalter 3310 output, wash water 3020, and hydroxyacid additive 3030 to be created based upon the specific characteristics of the crude oil supply 3010 to be desalted. The controllable fluid mixer 3350 is controlled by the controller 3110 to create and maintain the proper emulsion mix of crude oil 3010, wash water 3020, and hydroxyacid additive 3030.

The desalting mechanism 3000 of the present invention also includes a wash water supply 3020 and a hydroxyacid additive supply 3030 for supplying water-soluble hydroxyacid. In the embodiment of FIG. 3, as is preferred, the hydroxyacid additive 3030 is mixed with the wash water 3020 by the controllable fluid mixer 3040 before the crude oil/wash water emulsion is formed. Alternatively, the hydroxyacid additive 3030 could be mixed with the wash water 3020 and crude oil 3010 during the emulsion creation or after emulsion creation. The fluid mixer 3040 is controlled by the controller 3110 to create and maintain the proper solution mixture of hydroxyacid additive 3030 and wash water 3020. The hydroxyacid additive 3030 can be selected from the group consisting of glycolic acid, gluconic acid, C.sub.2-C.sub.4 alpha-hydroxy acids, malic acid, lactic acid, poly-hydroxy carboxylic acids, thioglycolic acid, chloroacetic acid, polymeric forms of the above hydroxyacids, poly-glycolic esters, glycolate ethers, and ammonium slat and alkali metal salts of these hydroxyacids, and mixtures thereof.

After mixing the solution of hydroxyacid additive 3030 and wash water 3020 with the controllable fluid mixer 3040, the resulting solution is input to a controllable flow control valve 3090 which is used to allow samples of the mixed hydroxyacid additive 3030 and wash water 3020 solution to be measured at a measurement station 3200. Measurements made on the solution samples would include but not be limited to solution pH, solution impurity levels, and percentage of hydroxyacid additive 3030 to wash water 3020. This information is sent to the controller 3110.

After mixing the solution of hydroxyacid additive 3030 and wash water 3020 with the controllable fluid mixer 3040, the resulting solution is also input to a controllable pump 3050 whose output is connected to a controllable flow control valve 3060. The controllable pump 3050 and the flow control valve 3060 work in conjunction under the command of the controller 3110 to control and maintain the wash water/hydroxyacid solution feed rate and pressure. The output of the flow control valve 3060 is an input to the controllable fluid mixer 3350 where the second emulsion of electrostatic desalter 3310 output, wash water 3020, and hydroxyacid additive 3030 is formed.

After mixing the second emulsion in the controllable fluid mixer 3350, the second emulsion passes through a controllable flow control valve 3360 before entering the electrostatic desalter 3170. The controllable flow control valve 3360, under command of the controller 3110, controls the flow rate of the second emulsion into the electrostatic desalter 3170 as well as allowing samples of the emulsion to be directed to the measurement station 3200. Measurements made on the solution samples would include but not be limited to impurity levels, temperature, amount of hydroxyacid additive 3030 and wash water 3020 solution, etc. This information is sent to the controller 3110.

The electrostatic desalter 3170 includes a liquid level sensor (LS) 3210 used to measure the aqueous level in the electrostatic desalter 3170. In the embodiment of FIG. 3, the measurement output of the liquid level sensor 3210 is routed to the controller 3110. The controller 3110 uses the liquid level measurement data to control the controllable flow control valve 3220 to recycle the effluent from the electrostatic desalter 3170 and control the aqueous layer and emulsion layer within the electrostatic desalter 3170. Alternatively, the liquid level sensor 3210 output may be directly connected to a level control valve instead of the controllable flow control valve 3220 to recycle the effluent. The controllable flow control valve 3220 along with the controllable pump 3370, under command of the controller 3110, control and maintain the recycled effluent flow rate and pressure to the controllable mixer 3380. The controllable flow control valve 3220 is also configured to allow samples of the effluent solution to be measured at a measurement station 3200. Measurements made on the solution samples would include but not be limited to solution pH, solution impurity levels, temperature, and amount of residual oil present in the effluent. This information is sent to the controller 3110.

The electrical power supply 3150 provides the voltage necessary to create the electric field necessary for electrostatic coalescence in the electrostatic desalter 3170. The controller 3110 controls the electrical power supply 3150 output. The electrical power supply 3150 output may be static (i.e. constant voltage with a current limit) or, preferably, able to change key parameters to enhance the desalting operation. The electrical power supply 3150 under the control of the controller 3110 would preferably be able to alter its' output to include but not be limited to changes in the voltage level applied to the electrostatic desalter 3170, the voltage waveform applied to the electrostatic desalter 3170, current limits (if any) on the electrical power supply 3150, or any combination thereof.

The desalted crude output of the electrostatic desalter 3170 passes through a pressure control valve 3180 and a controllable flow control valve 3190. The controllable flow control valve 3190 has two outputs to direct the desalted crude oil. Under control of the controller 3110, the controllable flow control valve 3190 controls and maintains the flow rate of desalted crude oil to the remaining refinery operations. Additionally, under control of the controller 3110, the controllable flow control valve 3190 can also direct samples of the desalted crude to the measurement station 3200. Measurements made on the solution samples would include but not be limited to impurity levels, temperature, residual hydroxyacid additive 3030 and wash water 3020 solution, etc. This information is sent to the controller 3110.

In the embodiment of FIG. 3, the controller 3110 takes measurements including but not limited to the various points described herein to evaluate the efficiency of the desalting mechanism 3000. Based upon the type of crude oil being processed, the controller 3110 can adjust various factors of the desalting operation including but not limited to the following:

The crude oil supply 3010 feed rate through the controllable pump 3070 and controllable flow control valve 3120

The temperature of the crude oil supply 3010 through the controllable heater 3130.

Control of the electrostatic desalter 3310 aqueous level and emulsion layers through the liquid level sensor 3340, the controllable flow control valve 3330, and the controllable flow control valve 3360.

The electrostatic desalter 3310 electric field through the controllable power supply 3300.

The solution mixture of hydroxyacid additive 3030 and wash water 3020 through the controllable fluid mixer 3040.

The flow rate of the solution mixture of hydroxyacid additive 3030 and wash water 3020 through the controllable pump 3050 and controllable flow control valve 3060.

The first emulsion formation of crude oil supply 3010 and recycled effluent from electrostatic desalter 3170 through controllable flow control valve 3220, controllable pump 3370, and controllable fluid mixer 3380.

The second emulsion formation through controllable fluid mixer 3350.

The electrostatic desalter 3170 electric field through the controllable electrical power supply 3150.

Control of the electrostatic desalter 3170 water level and emulsion layers through the liquid level sensor 3210, the controllable flow control valve 3220, and the controllable flow control valve 3190.

As different crude oils are processed by the desalting mechanism 3000, the characteristics necessary to efficiently desalt the crude oil will require some adjustment. Additionally, differences in electrostatic desalter 3310 and 3170 characteristics, wash water supply 3020 purity, etc between different desalting mechanisms 3000 require the storage of different control settings. The memory/data storage 3100 function of the desalting mechanism 3000 allows the controller to access and update, if required, the control settings required to efficiently process various types of crude oil supplies 3010. Preferably, the control settings are determined based upon maximizing the economic benefit for the desalting the crude oil supply 3010.

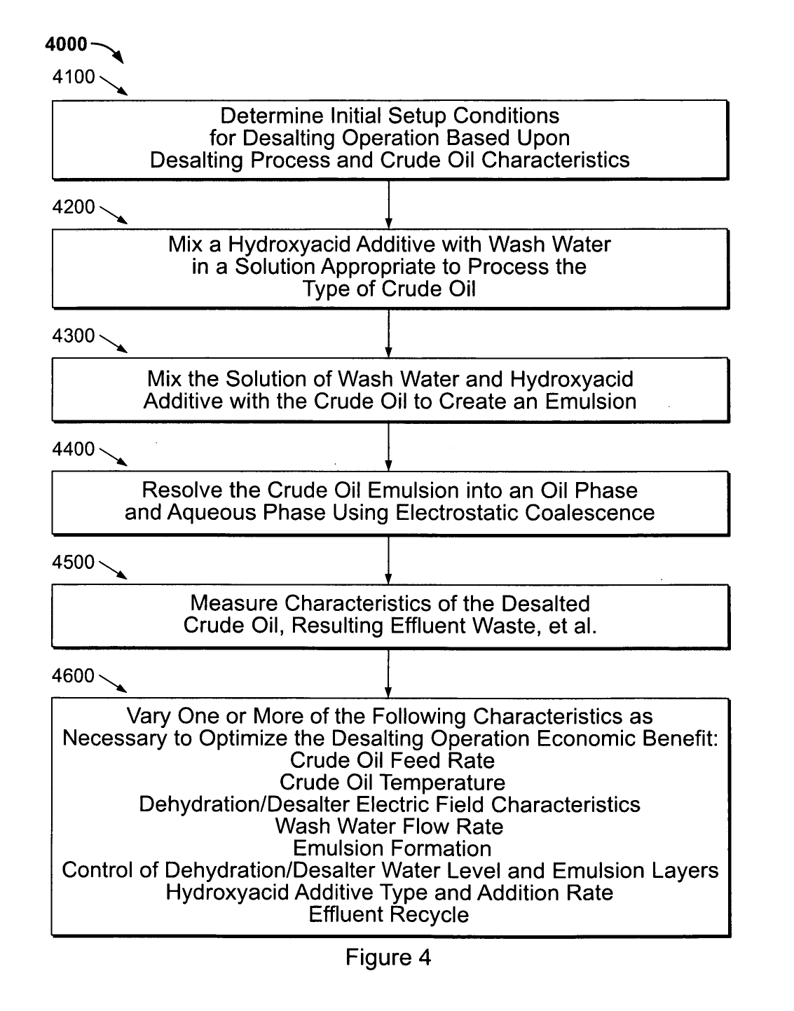

FIG. 4 shows a process diagram 4000 of one embodiment of the method of the present invention for a typical crude oil electrostatic desalting operation. The desalting process is set to an initial state in step 4100 based upon the characteristics of the configuration of the desalting operation and the characteristics of the crude oil to be desalted. A hydroxyacid additive is mixed with wash water in step 4200. The resulting hydroxyacid additive/wash water solution is then mixed with the crude oil to be desalted to create an emulsion in step 4300. The emulsion is resolved into a hydrocarbon or oil phase and an aqueous or water phase in step 4400. The characteristics of the effluent waste water, the desalted crude oil, or other points in the desalting operation that may or may not be dependent upon desalting configuration are measured in step 4500. Characteristics of the desalting operation to include but not limited to one or more of the following may be varied in step 4600 to maximize the economic benefit of the desalting operation based upon the measurements in step 4500: the crude oil feed rate, the crude oil temperature, the electric field characteristics of the dehydration/desalter mechanisms, the wash water flow rate, the crude oil emulsion formation, control of the dehydration/desalter water levels and emulsion layers, the hydroxyacid additive type and addition rate, and the effluent recycle (as appropriate).

The above embodiments are merely preferred and the scope of the invention defined by the claims below.

What is claimed is:

1. A method for removing calcium, other metals, and amines from crude oil in a refinery desalting process comprising the steps of:
adding a wash water to the crude oil;
adding the wash water to the crude oil to create an emulsion;
adding to the wash water or the emulsion at least one water-soluble hydroxyacid selected from the group consisting of glycolic acid, gluconic acid, C.sub.2-C.sub.4 alpha-hydroxy acids, malic acid, lactic acid, poly-hydroxy carboxylic acids, thioglycolic acid, chloroacetic acid, polymeric forms of the above hydroxyacids, poly-glycolic esters, glycolate ethers, and ammonium slat and alkali metal salts of these hydroxyacids, and mixtures thereof;
heating at least one of the crude oil, the wash water or the emulsion to a desired temperature;
resolving the emulsion containing the water-soluble hydroxyacid into a hydrocarbon phase and an aqueous phase using electrostatic coalescence, the metals and amines being transferred to the aqueous phase;
measuring a concentration of the metal or amine impurities in the hydrocarbon and/or aqueous phase; and
altering a characteristic of the desalting process to maintain residual impurity levels within the desalted crude as a function of the measured concentration.

2. The method as recited in claim 1 wherein the pH of the wash water, if the hydroxyacid is added to the wash water, is below 6.

3. The method as recited in claim 1 wherein the residual impurity levels are maintained below a setpoint.

4. The method as recited in claim 1 wherein the metal or amine impurities are measured in the hydrocarbon phase.

5. The method as recited in claim 1 wherein the metal removed is calcium and the concentration measured is a concentration of calcium.

6. The method as recited in claim 1 wherein the hydroxyacid selected is malic acid.

7. The method as recited in claim 1 wherein the characteristic is a temperature of the crude oil.

8. The method as recited in claim 1 wherein the characteristic is a crude oil supply feed rate.

9. The method as recited in claim 1 wherein the characteristic is a temperature of the emulsion.

10. The method as recited in claim 1 wherein the characteristic is the percentage of hydroxyacid additive to the wash water.

11. The method as recited in claim 1 wherein the characteristic is a flow rate of a solution mixture of hydroxyacid additive and wash water.

12. The method as recited in claim 1 wherein the characteristic is an electrostatic desalter electric field.

13. The method as recited in claim 1 wherein the characteristic is an electrostatic desalter water or emulation level.

14. The method as recited in claim 1 wherein the crude oil is heated.

15. The method as recited in claim 1 wherein the emulsion is heated.

16. A method for removing calcium, other metals, and amines from crude oil in a refinery desalting process consisting of:
adding a wash water to the crude oil;
adding the wash water to the crude oil to create an emulsion;
adding to the wash water or the emulsion at least one water-soluble hydroxyacid selected from the group consisting of glycolic acid, gluconic acid, C.sub.2-C.sub.4 alpha-hydroxy acids, malic acid, lactic acid, poly-hydroxy carboxylic acids, thioglycolic acid, chloroacetic acid, polymeric forms of the above hydroxyacids, poly-glycolic esters, glycolate ethers, and ammonium slat and alkali metal salts of these hydroxyacids, and mixtures thereof;
heating at least one of the crude oil, the wash water or the emulsion to a desired temperature;
resolving the emulsion containing the water-soluble hydroxyacid into a hydrocarbon phase and an aqueous phase using electrostatic coalescence, the metals and amines being transferred to the aqueous phase;
measuring a concentration of the metal or amine impurities in the hydrocarbon and/or aqueous phase; and
altering a characteristic of the desalting process to maintain residual impurity levels within the desalted crude as a function of the measured concentration.

17. A crude oil refinery operating the method as recited in claim 1.

18. An electrostatic desalter comprising;
a crude oil supply for storing crude oil;
a wash water supply for supplying wash water to the crude oil to form an emulsion;
a water-soluble hydroxyacid supply for supplying water-soluble hydroxyacid to the wash water or the emulsion;
a heater for changing the temperature of the crude oil, the wash water or the emulsion;
pumps and valves for controlling fluid flow in the desalting process; and
a controller for monitoring, controlling, and varying a characteristic of the desalting operation as a function of the concentration of metal and/or amine impurities in the aqueous phase and/or the hydrocarbon phase.
